(19) United States
(12) Patent Application Publication
(10) Pub. No.: US 2018/0134652 A1
(43) Pub. Date: May 17, 2018

(54) METHOD FOR PURIFYING AN ESTER COMPOUND

(71) Applicant: OCI COMPANY LTD., Seoul (KR)

(72) Inventors: Seong Hwan LEE, Seongnam-si (KR); Sang Hoon KIM, Seongnam-si (KR); Kyung Yeol KIM, Seongnam-si (KR); Yong Il KIM, Seongnam-si (KR)

(21) Appl. No.: 15/719,987

(22) Filed: Sep. 29, 2017

(30) Foreign Application Priority Data
Nov. 14, 2016 (KR) 10-2016-0151356

(51) Int. Cl. C07C 67/56; C07C 67/60; B01J 20/14; B01J 20/12; B01D 15/00; B01D 37/02

(52) U.S. Cl. CPC C07C 67/56; C07C 67/60; B01D 37/025; B01J 20/12; B01D 15/00; B01J 20/14

(57) ABSTRACT

Disclosed is a method for purifying an ester compound, including: adding a base to a resultant reaction mixture comprising a plasticizer of an ester compound synthesized by an esterification reaction of an acid and an alcohol so as to neutralize unreacted acids;
applying steam to the resultant reaction mixture which has been neutralized;
adding diatomite to the resultant reaction mixture which has been steamed, prior to filtration through a filter, and then subjecting the resultant reaction mixture to which the diatomite has been added to a primary filtration; and
adding an activated white clay to the resultant reaction mixture, and then subjecting the resultant reaction mixture which has been primarily filtrated to a secondary filtration through a filter.

### CROSS-REFERENCE TO RELATED APPLICATIONS

This application claims the priority of Korean Patent Application No. 10-2016-0151356 filed on Nov. 14, 2016, in the Korean Intellectual Property Office, the disclosure of which is hereby incorporated by reference in its entirety.

### BACKGROUND

### 1. Technical Field

The present disclosure relates to a method for purifying an ester compound.

### 2. Description of the Related Art

In general, the preparation of plasticizer largely includes reaction process, de-alcoholizing process for removing an excess of alcohol, purification process for removing impurities, or filtration process. Acidic catalyst such as PTSA (p-toluene sulfonic acid) or metallic catalyst such as TIPT (tetraisopropyl titanate) may be used as a reaction catalyst. The purification process is a final stage for the production of finished plasticizer in the preparation of plasticizer. During this process, unreacted materials, metallic ions, neutralized salts, catalytic salts, and the like are removed. In some cases, rinsing process for several times of water washing may be applied to remove impurities generated after neutralization process, prior to the purification process. This may cause problems of treating a large amount of wastewater and increased process time.

### SUMMARY

It is an aspect of the present disclosure to provide a method for purifying an ester compound capable of obtaining a product of a plasticizer of an ester compound from which impurities have been effectively removed without resorting to a rinsing process.

In one embodiment of the present disclosure, there is provided a method for purifying an ester compound, including:

adding a base to a resultant reaction mixture comprising a plasticizer of an ester compound synthesized by an esterification reaction of an acid and an alcohol so as to neutralize unreacted acids;

applying steam to the resultant reaction mixture which has been neutralized;

adding diatomite to the resultant reaction mixture which has been steamed, prior to filtration through a filter, and then subjecting the resultant reaction mixture to which the diatomite has been added to a primary filtration; and

adding an activated white clay to the resultant reaction mixture, and then subjecting the resultant reaction mixture which has been primarily filtrated to a secondary filtration through a filter.

The acid may include at least one selected from the group consisting of phthalic acid, terephthalic acid, and combinations thereof.

The alcohol may be an alkanol having a linear or branched (C₄-C₁₃) alkyl group.

The alcohol may include at least one selected from the group consisting of butanol, 2-ethylhexanol, isononyl alcohol, isodecyl alcohol, propyl heptanol, and combinations thereof.

The base may include at least one selected from the group consisting of sodium hydroxide (NaOH), sodium carbonate (Na₂CO₃), sodium bicarbonate (NaHCO₃), and combinations thereof.

The steam may be at a temperature of 140 to 200° C. and under a pressure of 2 bar to 10 bar.

The diatomite may have a pH of 7 to 10 and include 90 to 100 parts by weight of SiO₂, 1 to 10 parts by weight of Al₂O₃, 1 to 10 parts by weight of Fe₂O₃, 0.1 to 1 part by weight of CaO, 0.1 to 1 part by weight of MgO, and water.

The activated white clay may have a free acidity of 1 to 10 mg KOH/g, and 90 to 100% by weight of particle sizes may be 200 mesh.

The activated white clay may include 65 to 75% by weight of SiO₂, 10 to 20% by weight of Al₂O₃, 1 to 2% by weight of Fe₂O₃, 0.1 to 3% by weight of CaO, 0.1 to 2% by weight of MgO, and 0.1 to 10% by weight of water.

The activated white clay may be added in an amount of 1 to 10 parts by weight based on 100 parts by weight of the primarily filtrated resultant reaction mixture.

The plasticizer of a product of the ester compound obtained by the method of purifying the ester compound has a high purity with a low impurity content, and the plasticizer of a product obtained with the high purity ester compound has a high liquid volume resistivity (LVR) and an improved APHA COLOR, which are useful for being applied as a plasticizer.

### BRIEF DESCRIPTION OF THE DRAWINGS

FIG. 1 is a schematic diagram illustrating a method of purifying ester compounds according to one embodiment of the present invention.

### DETAILED DESCRIPTION

Hereinafter, exemplary embodiments of the present disclosure will be described in detail with reference to the accompanying drawings, so that they can readily be practiced by those skilled in the art to which the present disclosure pertains. The present disclosure may be embodied in many different forms and is not limited to the embodiments described herein.

In one embodiment of the present disclosure, there is provided a method for purifying an ester compound, including:

adding a base to a resultant reaction mixture comprising a plasticizer of an ester compound synthesized by an esterification reaction of an acid and an alcohol so as to neutralize unreacted acids;

applying steam to the resultant reaction mixture which has been neutralized;

adding diatomite to the resultant reaction mixture which has been steamed, prior to filtration through a filter, and then subjecting the resultant reaction mixture to which the diatomite has been added to a primary filtration; and

adding an activated white clay to the resultant reaction mixture, and then subjecting the resultant reaction mixture which has been primarily filtrated to a secondary filtration through a filter.

The resultant reaction mixture comprising an ester compound synthesized by esterification reaction of an acid and an alcohol may be obtained in the form of a mixture of unreacted materials, metallic ions, neutralized salts, and catalytic salts, together with the synthesized ester compounds, such that the resultant reaction mixture may contain impurities.

When the content of the impurities in the product of the plasticizer of the finally obtained ester compound is increased, the ester compounds are inhibited from manifesting various physical properties required as a plasticizer.

For example, PVC wire coating may use a plasticizer as one of additives and a plasticizer of ester compound may be used for the plasticizer. It is important that the plasticizer has a high liquid volume resistivity (LVR), since the PVC wire coating should have a high resistance value to ensure good insulation. The plasticizer of ester compound obtained by the method of purifying ester compounds can increase the liquid volume resistivity by lowering the content of impurities and is suitable for use as a plasticizer for PVC wire coating.

In addition, the plasticizer of the ester compound obtained by the method of purifying ester compounds has a low APHA COLOR value.

Hereinafter, each step of the method of purifying ester compounds will be described in detail.

The method can be effectively applied to ester compounds produced by an ester exchange reaction of acids and alcohols.

The acid may include at least one selected from the group consisting of phthalic acid, terephthalic acid, and combinations thereof.

The alcohol may be an aliphatic alcohol, specifically, an alkanol having a linear or branched (C₄-C₁₃) alkyl group.

For examples, the alcohol may include at least one selected from the group consisting of butanol, 2-ethylhexanol, isononyl alcohol, isodecyl alcohol, propyl heptanol, and combinations thereof.

The resultant reaction mixture contains impurities as described above, and in particular, unreacted acids, which must be necessarily neutralized. Thus, base may be added to the resultant reaction mixture to neutralize the unreacted acids.

The base, for example, may include, but is not limited to, at least one selected from the group consisting of sodium hydroxide (NaOH), sodium carbonate (Na₂CO₃), sodium bicarbonate (NaHCO₃), and combinations thereof.

The base may be added in an amount of 0.1 to 1 part by weight based on 100 parts by weight of the resultant reaction mixture which has been neutralized. Within the above range of contents, the pH of the resultant reaction mixture can be suitably controlled.

Specifically, the resultant reaction mixture which has been neutralized may have a pH of 7 to 10.

When high-temperature and high-pressure steam is applied to the resultant reaction mixture having been subjected to the neutralization treatment, alcohol is removed by azeotropic distillation together with the steam. In addition to removing the unreacted alcohol by the steam treatment step, Ti-based catalyst may be reacted with water and be extracted into solid state TiO₂, and the TiO₂ may be removed by a subsequent filtration step.

The high-temperature and high-pressure steam may be at a temperature of 140 to 200° C. and under a pressure of 2 bar to 10 bar.

Then, diatomite may be added to the steamed resultant reaction mixture, and subsequently the resultant reaction mixture to which the diatomite has been added was subjected to a primary filtration.

The added diatomite may be directly mixed with the resultant reaction mixture to allow the impurities to be adsorbed. The diatomite to which the impurities particles are adsorbed may be filtrated and removed through a filter in a primary filtration step.

The content ratio of the diatomite may be appropriately controlled depending on detailed characteristic specifications thereof. Specifically, the diatomite added to the resultant reaction mixture which has been steamed may be added in an amount of 1 to 10 parts by weight based on 100 parts by weight of the resultant reaction mixture which has been steamed. The effect of removing the impurities can be further improved by adding the diatomite within the above amount.

Further, the filter may include diatomite. Specifically, the filter may be a filter membrane on which a precoat layer containing diatomite is formed.

The filter membrane may be that formed of a known material such as polypropylene, polytetrafluoroethylene (PTFE), polyester, or acrylic material, but is not limited thereto.

The diatomite added and the diatomite forming the precoat layer have a porous structure containing silica as a main component and have a melting point of 1400° C. or more and is stable to general chemical action.

The diatomite improves filtration rate, protects the filter, and in particular, effectively removes the neutralized salts generated from the previously performed neutralization process.

Diatomite is a kind of fossil soil that is a collection of debris from single-celled plants called diatoms, deposited on a sea-bed and a bottom of lake. Diatoms have the ability to extract silica from underwater environments to form their own rigid skeletal structure, wherein diatomaceous sedimentary rocks formed by sedimentation of diatoms skeleton under the influence of diagenesis are mostly composed of amorphous silica and are produced in a large scale aggregation state. Diatomite is classified as fresh water type and sea water type depending on the places where it is deposited, and also shows different physical properties.

Specifically, the diatomite may have a pH value of 7 to 10.

Specifically, the diatomite may include 90 to 100 parts by weight of SiO₂, 1 to 10 parts by weight of Al₂O₃, 1 to 10 parts by weight of Fe₂O₃, 0.1 to 1 part by weight of CaO, 0.1 to 1 part by weight of MgO, and a small quantity of water.

Then, an activated white clay may be added to the resultant reaction mixture which has been primarily filtrated, and then subjected to a secondary filtration through a filter.

The added activated white clay may be directly mixed with the resultant reaction mixture to allow the impurities to be adsorbed. The activated white clay to which the impurities particles are adsorbed may be filtrated and removed through a filter in a secondary filtration step.

The activated white clay added to the resultant reaction mixture which has been primarily filtrated may be added in an amount of 1 to 10 parts by weight based on 100 parts by weight of the resultant reaction mixture which has been primarily filtrated. The effect of removing the impurities can be further improved by adding the activated white clay within the above amount.

Further, the filter used in the primary filtration step may be used as it is.

The added activated white clay is a substance which can be obtained, for example, by acid treatment of acidic white clay, clay, or the like using sulfuric acid or hydrochloric acid. Acid treatment of acidic white clay, clay, etc., removes free alumina and other unnecessary materials, resulting in the formation of porous activated white clay. The activated white clay has improved activity, and, for example, has better adsorption and decolorizing ability than acidic white clay.

The activated white clay improves filtration rate, protects a filter, and in particular, effectively removes neutralized salts generated from the previously performed neutralization process.

The activated white clay can effectively work to remove impurities from phthalic acid plasticizer and terephthalic acid plasticizer.

The activated white clay may specifically include 65 to 75% by weight of SiO₂, 10 to 20% by weight of Al₂O₃, 1 to 2% by weight of Fe₂O₃, 0.1 to 3% by weight of CaO, 0.1 to 2% by weight of MgO, and 0.1 to 10% by weight of water.

The activated white clay may have a free acidity of 1 to 10 mg KOH/g, and 90 to 100% by weight of particle sizes may be 200 mesh.

The method of purifying ester compounds may be carried out in two step: a filtration step of using diatomite and a post-treatment filtration step of using activated white clay.

By using diatomite and activated white clay in order as a filter aid, the activated white clay can effectively act on impurities that cannot be removed by the diatomite, and in particular metallic impurities can be effectively removed. Since the metallic impurities are effectively removed, the plasticizer of the ester compound finally obtained has a high liquid volume resistivity (LVR).

In addition, since the activated white clay has a high adsorption decolorizing ability, the APHA COLOR of the product of the plasticizer of the ester compound finally obtained is improved.

Generally, in the case of rinsing-free process, since the impurities are not removed by water, it is necessary to remove fine particles such as neutralized salts of unreacted terephthalates, inorganic salts and organic salts through a filtration filter, which may cause process speed of the filter to slow or filter clogging. Impurities such as neutralized salts of unreacted terephthalates, catalytic salts, inorganic salts and organic salts have a very small crystal grain sizes and easily form a film on the filter surface, which in turn easily cause problems of filter clogging.

Since the method of purifying the ester compound, even as a rinsing-free process, can effectively remove metallic impurities such as neutralized salts and catalytic salts by performing a two-stage filtration process by sequentially using the diatomite and the activated white clay together, the method of purifying the ester compound is able to improve the filtration speed and efficiency of the filter. Since the method of purifying the ester compound possibly shortens the filtration time as it improves the filtration rate, it possibly reduces the production process time of the ester compounds.

The plasticizer of the ester compound obtained by the method of purifying ester compounds may have a concentration of impurities ranging from several ppm to several hundred ppm.

Hereinafter, specific examples of the present disclosure will be described. It is to be understood, however, that the examples described below are only for illustrative purposes or to illustrate the present disclosure, and the present disclosure should not be construed as being limited thereto.

### EXAMPLES

Hereinafter, in the examples, Na ion content, liquid volume resistivity (LVR) and APHA COLOR values were measured as follows.

(1) Na Ion Content

Na ion content was measured with a metal ion analyzer ICP (manufactured by Agilent, 720 ICP-OES model).

However, the detection limit of Na content is 0.50 ppm.

(2) Liquid Volume Resistivity (LVR)

LVR was measured with a volume resistance meter (manufactured by HIOKI, SME series model).

(3) APHA COLOR Value

According to ASTM D 1209-93, the APHA COLOR value was visually observed using a Pt/Co standard reagent.

### Example 1

350 g (2.107 moles) of terephthalic acid, 687 g (5.28 moles) of 2-ethylhexanol and 0.208 g (200 ppm) of tetraisopropoxide were added to a 2 L round bottom flask equipped with a stirrer, thermometer, heating mantle and distillation column. Upon heating, the reaction initiated at about 180° C. As the reaction progressed, produced water was generated and the produced water was removed by the distillation column. After completion of the reaction, aqueous Na₂CO₃ solution was added to neutralize unreacted terephthalates, and steam was added at 180° C. to remove the remaining 2-ethylhexanol. N₂ was then injected to remove a small amount of water, and vigorously stirred with 5 g of diatomite, followed by filtration with a vacuum filter. The diatomite had a pH of 10 and comprises 90 to 100 parts by weight of SiO₂, 1 to 10 parts by weight of Al₂O₃, 1 to 10 parts by weight of Fe₂O₃, 0.1 to 1 part by weight of CaO, 0.1 to 1 part by weight of MgO, and water.

Then, for the resulting resultant reaction mixture containing dioctyl terephthalate (DOTP) obtained by an ester reaction of terephthalic acid and 2-ethylhexanol, the Na ion content was 0.84 ppm, the APHA COLOR value was 25, and the liquid volume resistivity (LVR) was 2.80×10¹¹ Ω·cm.

1 g of diatomite and 1 g of activated white clay were added to the resultant reaction mixture containing dioctyl terephthalate (DOTP) obtained above, followed by stirring at 100° C. for 30 minutes. The reaction solution was filtrated using a vacuum filtration apparatus. After filtration, the liquid volume resistivity of the resulting product containing dioctyl terephthalate (DOTP) was measured as 48.0×10¹¹ Ω·cm, the Na ion content was less than or equal to 0.50 ppm, and the APHA COLOR value was measured as 20.

### Example 2

350 g (2.107 moles) of terephthalic acid, 687 g (5.28 moles) of 2-ethylhexanol and 0.208 g (200 ppm) of tetraisopropoxide were added to a 2 L round bottom flask equipped with a stirrer, thermometer, heating mantle and distillation column. Upon heating, the reaction initiated at about 180° C. As the reaction progressed, produced water was generated and the produced water was removed by the distillation column. After completion of the reaction, aqueous Na₂CO₃ solution was added to neutralize unreacted terephthalates, and steam was added at 180° C. to remove the remaining 2-ethylhexanol. N₂ was then injected to remove a small amount of water, and vigorously stirred with 5 g of diatomite, followed by filtration with a vacuum filter. The diatomite had a pH of 10 and comprised 90 to 100 parts by weight of SiO₂, 1 to 10 parts by weight of Al₂O₃, 1 to 10 parts by weight of Fe₂O₃, 0.1 to 1 part by weight of CaO, 0.1 to 1 part by weight of MgO, and water.

Then, for the resultant reaction mixture containing dioctyl terephthalate (DOTP) obtained by an ester reaction of terephthalic acid and 2-ethylhexanol, the Na ion content was 0.84 ppm, the APHA COLOR value was 25, and the liquid volume resistivity (LVR) was 2.80×10¹¹ Ω·cm.

2 g of activated white clay was added to the resultant reaction mixture containing dioctyl terephthalate (DOTP) obtained above, followed by stirring at 100° C. for 30 minutes. The reaction solution was filtrated using a vacuum filtration apparatus. After filtration, the liquid volume resistivity of the resulting product containing dioctyl terephthalate (DOTP) was measured as 69.9×10¹¹ Ω·cm, the Na ion content was less than or equal to 0.50 ppm, and the APHA COLOR value was measured as 15.

### Comparative Example 1

350 g (2.107 moles) of terephthalic acid, 687 g (5.28 moles) of 2-ethylhexanol and 0.208 g (200 ppm) of tetraisopropoxide were added to a 2 L round bottom flask equipped with a stirrer, thermometer, heating mantle and distillation column. Upon heating, the reaction initiated at about 180° C. As the reaction progressed, produced water was generated and the produced water was removed by the distillation column. After completion of the reaction, aqueous Na₂CO₃ solution was added to neutralize unreacted terephthalates, and steam was added at 180° C. to remove the remaining 2-ethylhexanol. N₂ was then injected to remove a small amount of water, and vigorously stirred with 5 g of diatomite, followed by filtration with a vacuum filter. The diatomite had a pH of 10 and comprised 90 to 100 parts by weight of SiO₂, 1 to 10 parts by weight of Al₂O₃, 1 to 10 parts by weight of Fe₂O₃, 0.1 to 1 part by weight of CaO, 0.1 to 1 part by weight of MgO, and water.

Then, for the resultant reaction mixture containing dioctyl terephthalate (DOTP) obtained by an ester reaction of terephthalic acid and 2-ethylhexanol, the Na ion content was 0.84 ppm, the APHA COLOR value was 25, and the liquid volume resistivity (LVR) was 2.80×10¹¹ Ω·cm.

The resultant reaction mixture containing dioctyl terephthalate (DOTP) obtained above was stirred at 100° C. for 30 minutes. The reaction solution was filtrated using a vacuum filtration apparatus. After filtration, the liquid volume resistivity of the resulting product containing dioctyl terephthalate (DOTP) was measured as 3.0×10¹¹ Ω·cm, the Na ion content was less than or equal to 0.84 ppm, and the APHA COLOR value was measured as 25.

### Comparative Example 2

350 g (2.107 moles) of terephthalic acid, 687 g (5.28 moles) of 2-ethylhexanol and 0.208 g (200 ppm) of tetraisopropoxide were added to a 2 L round bottom flask equipped with a stirrer, thermometer, heating mantle and distillation column. Upon heating, the reaction initiated at about 180° C. As the reaction progressed, produced water was generated and the produced water was removed by the distillation column. After completion of the reaction, aqueous Na₂CO₃ solution was added to neutralize unreacted terephthalates, and steam was added at 180° C. to remove the remaining 2-ethylhexanol. N₂ was then injected to remove a small amount of water, and vigorously stirred with 5 g of diatomite, followed by filtration with a vacuum filter. The diatomite had a pH of 10 and comprised 90 to 100 parts by weight of SiO₂, 1 to 10 parts by weight of Al₂O₃, 1 to 10 parts by weight of Fe₂O₃, 0.1 to 1 part by weight of CaO, 0.1 to 1 part by weight of MgO, and water.

Then, for the resultant reaction mixture containing dioctyl terephthalate (DOTP) obtained by an ester reaction of terephthalic acid and 2-ethylhexanol, the Na ion content was 0.84 ppm, the APHA COLOR value was 25, and the liquid volume resistivity (LVR) was 2.80×10¹¹ Ω·cm.

2 g of diatomite was added to the resultant reaction mixture containing dioctyl terephthalate (DOTP) obtained above, followed by stirring at 100° C. for 30 minutes. The reaction solution was filtrated using a vacuum filtration apparatus. After filtration, the liquid volume resistivity of the resulting product containing dioctyl terephthalate (DOTP) was measured as 9.32×10¹¹ Ω·cm, the Na ion content was less than or equal to 0.84 ppm, and the APHA COLOR value was measured as 25.

The results are summarized in Table 1 below.

TABLE 1

| Division | Ex. 1 | Ex. 2 | C. Ex. 1 | C. Ex. 2 |
|---|---|---|---|---|
| Post-treatment | Diatomite + | Activated | Not | Diatomite |
| filtration step | Activated | white clay | performed | |
| | white clay | | | |
| LVR | 48.0. | 69.9. | 2.80. | 9.32. |
| (10¹¹ Ω · cm) | | | | |
| Na content | 0.50 or less | 0.50 or less | 0.84. | Not varied |
| (ppm) | | | | |
| APHA | 20 | 15 | 25 | Not varied |
| COLOR | | | | |

The results shown in Table 1 indicates that the content of sodium ion, which is a metallic ion, in Examples 1 and 2, was lower than that of Comparative Examples 1 and 2, and the liquid volume resistivity was improved.

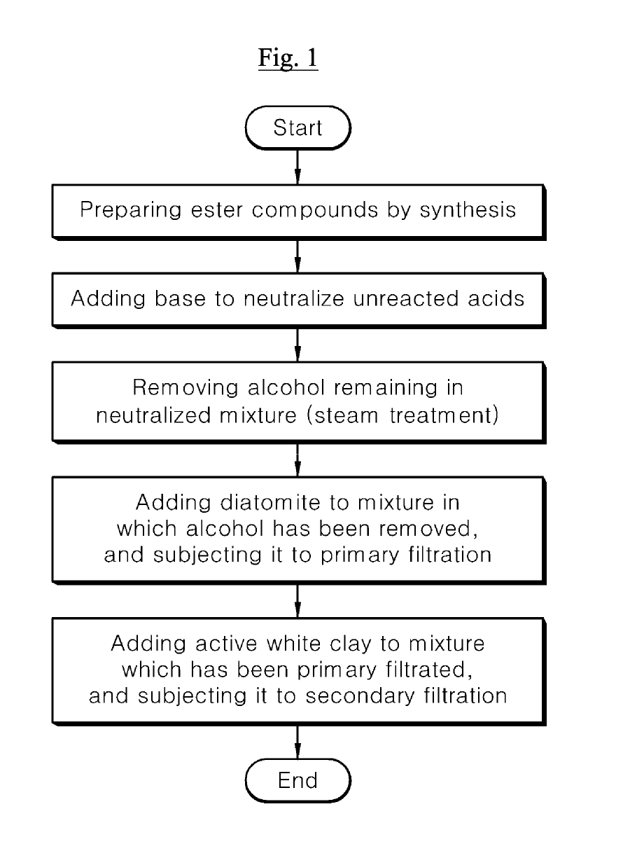

FIG. 1 is a schematic diagram illustrating a method of purifying ester compounds according to one embodiment of the present invention.

While the present disclosure has been particularly shown and described with reference to exemplary embodiments thereof, it is to be understood by those skilled in the art that the disclosure is not limited to the embodiments and various changes in form and details may be made therein without departing from the spirit and scope of the invention as defined by the appended claims.

What is claimed is:

1. A method of purifying ester compounds, comprising:
adding a base to a resultant reaction mixture comprising a plasticizer of an ester compound synthesized by an esterification reaction of an acid and an alcohol so as to neutralize unreacted acids;
applying steam to the resultant reaction mixture which has been neutralized;
adding diatomite to the resultant reaction mixture which has been steamed, prior to filtration through a filter, and then subjecting the resultant reaction mixture to which the diatomite has been added to a primary filtration; and
adding an activated white clay to the resultant reaction mixture, and then subjecting the resultant reaction mixture which has been primarily filtrated to a secondary filtration through a filter.

2. The method according to claim 1, wherein the acid comprises at least one selected from the group consisting of phthalic acid, terephthalic acid, and combinations thereof.

3. The method according to claim 1, wherein the alcohol is an alkanol having a linear or branched (C₄-C₁₃) alkyl group.

4. The method according to claim 1, wherein the alcohol comprises at least one selected from the group consisting of butanol, 2-ethylhexanol, isononyl alcohol, isodecyl alcohol, propyl heptanol, and combinations thereof.

5. The method according to claim 1, wherein the base comprises at least one selected from the group consisting of sodium hydroxide (NaOH), sodium carbonate (Na₂CO₃), sodium bicarbonate (NaHCO₃), and combinations thereof.

6. The method according to claim 1, wherein the steam is at a temperature of 140 to 200° C. and under a pressure of 2 bar to 10 bar.

7. The method according to claim 1, wherein the diatomite has a pH of 7 to 10 and comprises 90 to 100 parts by weight of SiO₂, 1 to 10 parts by weight of Al₂O₃, 1 to 10 parts by weight of Fe₂O₃, 0.1 to 1 part by weight of CaO, 0.1 to 1 part by weight of MgO, and water.

8. The method according to claim 1, wherein the activated white clay has a free acidity of 1 to 10 mg KOH/g, and 90 to 100% by weight of particle sizes are 200 mesh.

9. The method according to claim 1, wherein the activated white clay comprises 65 to 75% by weight of SiO₂, 10 to 20% by weight of Al₂O₃, 1 to 2% by weight of Fe₂O₃, 0.1 to 3% by weight of CaO, 0.1 to 2% by weight of MgO, and 0.1 to 10% by weight of water.

10. The method of purifying ester compounds according to claim 1, wherein the activated white clay is added in an amount of 1 to 10 parts by weight based on 100 parts by weight of the resultant reaction mixture which has been primarily filtrated.
